(19) United States
(12) Patent Application Publication
(10) Pub. No.: US 2011/0015742 A1
(43) Pub. Date: Jan. 20, 2011

(54) Spine fusion cage

(76) Inventor: Wei-Chen Hong, Tainan City (TW)

Correspondence Address: BACON & THOMAS, PLLC, 625 SLATERS LANE, FOURTH FLOOR, ALEXANDRIA, VA, 22314-1176, US

(21) Appl. No.: 12/591,183

(22) Filed: Nov. 12, 2009

(30) Foreign Application Priority Data
Jul. 20, 2009 (TW) 098124403

(51) Int. Cl. A61F 2/44

(52) U.S. Cl. 623/17.11

(57) ABSTRACT

A spine fusion cage comprises a housing, a movable member, a sliding member and a screw. The upper surface of the housing has an opening through which the movable member can move in a vertical direction. The sliding member is provided within the housing and can move in a longitudinal direction of the spine fusion cage. The movable member has an activation surface. In the case that the activation surface is in contact with the sliding member, movement of the sliding member in the longitudinal direction of the spine fusion cage causes the movable member to move up or down. The sliding member is moved by turning the screw such that the movable member can move up or down.

### BACKGROUND OF THE INVENTION

1. Field of the Invention

The present invention relates to a spine fusion cage, especially to a spine fusion cage which is expandable or height-adjustable.

2. Brief Description of Prior Art

In U.S. Pat. No. 5,665,122A, an expandable cage driven by a screw is disclosed.

In U.S. Pat. No. 5,665,122A, a cone body located at one end of a screw is moved toward a hole having an inner tapered surface by screwing the screw into a threaded hole such that the upper portion of the cage moves in a vertical direction with respect to the lower portion of the cage. According to the cage structure disclosed in U.S. Pat. No. 5,665,122A, the displacement stroke of the upper portion with respect to the lower portion of the cage never exceeds the diameter of the screw. Therefore, a sufficient displacement stroke cannot be provided.

In US Patent No. 2008/161933A, an expandable cage driven by fluid is disclosed. The cage disclosed in US Patent No. 2008/161933A comprises a multistage telescopic sleeve structure driven by fluid. Although the cage disclosed in US Patent No. 2008/161933A can provide a sufficient displacement stroke, the cage is not only complicated in its structure but also needs additional control equipment for operation. Thus, it is unfavorable in manufacturing, assembling and operation.

### SUMMARY OF INVENTION

The object of this invention is to provide a spine fusion cage which has a simple structure and is easy to be manufactured and assembled.

Another object of this invention is to provide a spine fusion cage which is capable of providing a sufficient displacement stroke.

Another object of this invention is to provide a spine fusion cage which can be operated in easy manner.

The above and other objects can be achieved by a spine fusion cage according to this invention, comprising a housing, a movable member, a sliding member and a screw, wherein the upper end of the housing has an opening through which the movable member can move in the vertical direction, the sliding member being provided within the housing and moving in the longitudinal direction of the spine fusion cage, the movable member having an activation surface to be in contact with the sliding member such that movement of the sliding member in the longitudinal direction of the spine fusion cage causes the movable member to move up or down, the sliding member being moved by rotating the screw.

The above and other objects and advantages of the present invention will become apparent from the following description with reference to the accompanying drawings which illustrate the example of the present invention.

### DETAILED DESCRIPTION OF PREFERRED EMBODIMENT

The technical features of the present invention will become more apparent from the detailed description of the preferred embodiment in conjunction with the accompanying drawings. The preferred embodiment is purely for descriptive purpose, not for limitation on the scope of the present invention. And the accompanying drawings are also for illustrative purpose, which may not be made according to the practical scale.

FIG. 1 is a perspective view showing the spine fusion cage, designated by 10, according to the present invention.

The spine fusion cage 10 according to this invention comprises a housing 11 and a movable member 12.

The upper surface of the movable member 12 and the lower surface of the housing 11 are regularly formed with surface structure of triangular wave shape to serve as skidproof structure. The surface structure can also be formed with irregular shape. Alternatively, the upper surface of the movable member 12 and the lower surface of the housing 11 can also be roughened so as to obtain the skidproof structure.

The housing 11 is substantially formed as rectangular shape. In the longitudinal direction of the spine fusion cage 10, the housing 11 has a front end 111 and a rear end 112 opposite to the front end 111. Preferably, the front end 111 is formed as a pyramid, cone, curved surface, semi-spherical shape or streamline shape, or the front end 111 is chamfered so as to facilitate insertion.

In order to arrange parts within the housing 11, the rear end 112 of the housing 11 is formed as a separate cover plate which is fixed on the housing 11 by fastening means such as screw, adhesive, snap-fit, welding, soldering and the like.

The upper end of the housing 11 has an opening 113 through which the movable member 12 can move in the vertical direction between an extended position and a retracted position with a controllable manner.

FIGS. 2 and 3 are sectional views perpendicular to the longitudinal direction of the spine fusion cage 10, in which the movable member 12 is located at the retracted position as shown in FIG. 2, and at the extended position in FIG. 3. Preferably, a first flange 121 having a width larger than the width of the opening 113 is formed at the lower end of the movable member 12 such that the movable member is prevented from completely extending out so as to prevent the movable member from escaping from the housing 11. Optionally, a second flange 122 can also be formed at the upper end of the movable member 12.

FIGS. 4 and 5 are vertical sectional views in the longitudinal direction of the spine fusion cage 10, in which the movable member 12 is located at the retracted position as shown in FIG. 4, and at the extended position in FIG. 5. The right bottom corner of the movable member 12 is cut off so as to form an activation surface 123 inclined with respect to the longitudinal direction. The activation surface 123 can be a plane or a curved surface.

As shown in FIG. 4, the sliding member 13 has a shape substantially corresponding to the cut off portion of the movable member 12. When the movable member 12 is located at the retracted position, the sliding member 13 approximately occupies the space of the cut of portion, and the inclined surface 131 of the sliding member 13 is in surface contact with the activation surface 123 of the movable member 12.

The screw 14 is screwed into the threaded hole 114 on the cover plate and engaged with the threaded hole 114 such that rotation of the screw 14 causes the screw 14 to move in the longitudinal direction with respect to the cover plate. In this manner, the sliding member 13 moves in the longitudinal direction by rotating the screw 14. With the movement of the sliding member 13 in the longitudinal direction, the movable member 12 is moved up or down correspondingly.

As shown in FIG. 5, when the sliding member 13 moves beyond the range of the activation surface 123, the movable member 12 is located at the extended position. At this moment, the sliding member 13 is located beneath the movable member 12 and abutted against the lower surface of the movable member 12 so as to firmly support the movable member 12 and prevent the movable member 12 from being retracted back. The displacement stroke of the movable member 12 is equivalent to the height of the sliding member 13.

According to this invention, the sliding member 13 is attached to one end of the screw 14 in such a manner that the sliding member 13 is rotatable with respect to the screw 14. Therefore, the sliding member 13 would not be rotated when the sliding member 13 is moved along with the screw 14. In other words, the sliding member 13 is moved in a constant orientation along with the screw 14 in the longitudinal direction. For example, the sliding member 13 has a blind hole 132 with a neck portion, while the screw 14 has a head 141 with an outer diameter slightly larger than the inner diameter of the neck portion such that the head 141 of the screw 14 is fitted into the blind hole 132 under plastic deformation of the neck portion of the blind hole 132 and/or the head 141 of the screw 14. In this manner, the sliding member 13 can be attached to the screw 14, and the sliding member is freely rotatable with respective to the screw 14, or the screw 14 is rotatable with respect to the sliding member 13. The structure for attachment of the sliding member 13 to the screw 14 is schematically shown in FIG. 6.

If it is not necessary to retract the movable member from the extended position, the abovementioned structure for attachment of the sliding member 13 to the screw 14 may be omitted. That is, the sliding member can be merely pushed by the screw but is unable to be pulled by the screw.

According to the present invention, at least one of the parts of the spine fusion cage is made by biocompatible material, which may be metal, alloy or polymer, such as Ti, Ti alloy and Polyetheretherketone (PEEK) and the like.

The spine fusion cage according to the present invention can be implanted between the vertebras of a patient so as to fuse the vertebra.

The spine fusion cage according to the present invention can be implanted into the collapsed vertebra caused by vertebral compression fracture as a support for vertebral reconstruction.

While this invention has been described with reference to the embodiment, it should be understood that various changes and modifications could be made within the spirit and scope of the inventive concepts described. Accordingly, it is intended that the invention shall not be limited to the disclosed embodiment but have the full scope permitted by the language of the following claims.

### BRIEF DESCRIPTION OF THE ACCOMPANYING DRAWINGS

FIG. 1 is a perspective view showing the spine fusion cage of the present invention;

FIGS. 2 and 3 are sectional views perpendicular to the longitudinal direction of the spine fusion cage of the present invention, in which the movable member is located at the retracted position as shown in FIG. 2, and at the extended position in FIG. 3;

FIGS. 4 and 5 are vertical sectional views in longitudinal direction of the spine fusion cage of the present invention, in which the movable member is located at the retracted position as shown in FIG. 4, and at the extended position in FIG. 5; and

FIG. 6 is a view showing the connection between the sliding member and the screw.

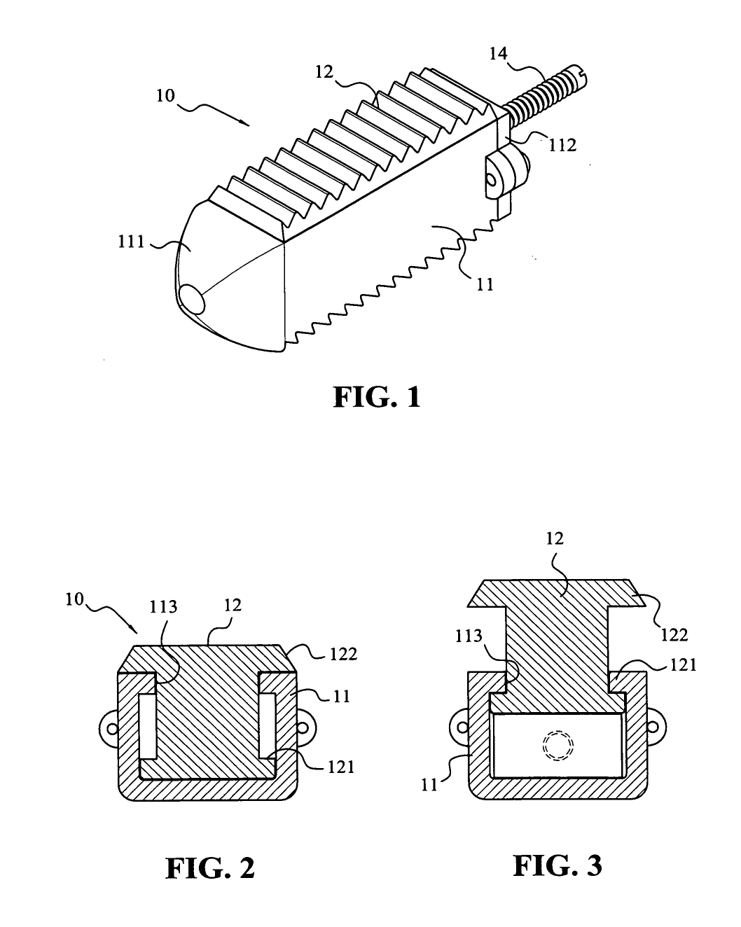

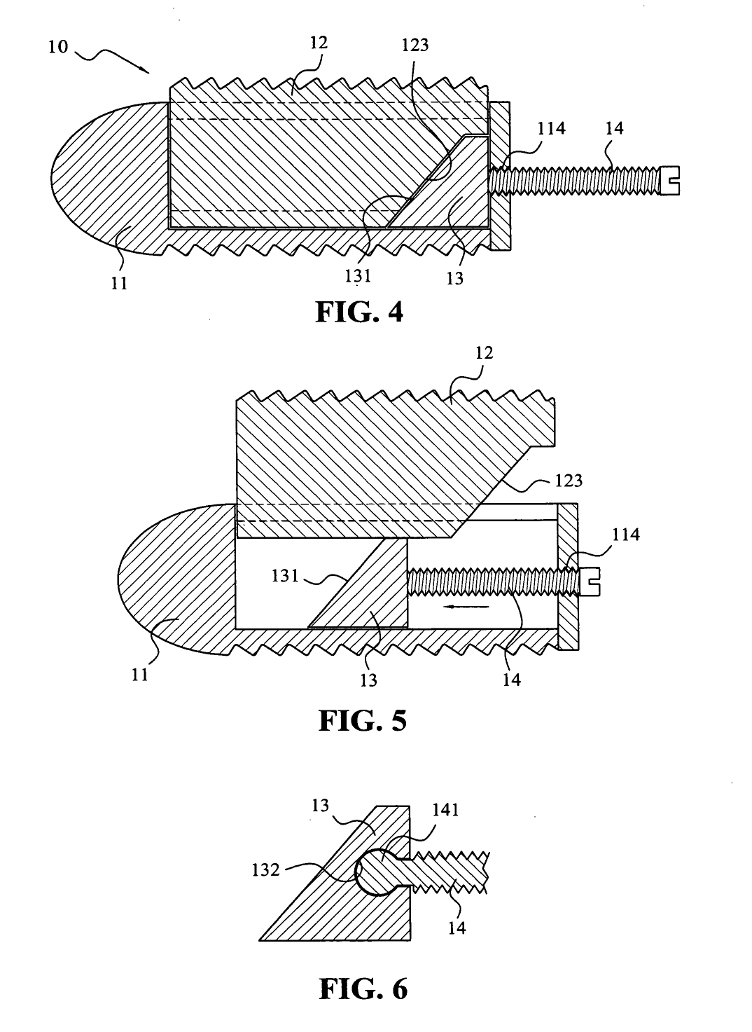

What is claimed is:

1. A spine fusion cage, comprising a housing, a movable member, a sliding member and a screw, wherein:
an upper side of said housing has an opening through which the movable member is movable in a vertical direction,
said sliding member is provided within said housing and movable in a longitudinal direction of said spine fusion cage,
said movable member has an activation surface, in the case that the activation surface is in contact with said sliding member, movement of said sliding member in the longitudinal direction of said spine fusion cage causes the movable member to move up or down,
said sliding member is moved by rotating the screw.

2. The spine fusion cage as claimed in claim 1, wherein said housing has a front end and a rear end, said screw is engaged with a threaded hole formed on said rear end such that said screw is moved in the longitudinal direction with respect to said rear end by rotating said screw so as to move said sliding member.

3. The spine fusion cage as claimed in claim 2, wherein said sliding member is attached to one end of said screw in such a manner that said sliding member is rotatable with respect to said screw.

4. The spine fusion cage as claimed in claim 1, wherein the movable member is provided with a first flange at a lower end of said movable member so as to prevent said movable member from escaping from said housing from said opening.

5. The spine fusion cage as claimed in claim 1, wherein said movable member is provided with a second flange provide at an upper end of said movable member.

6. The spine fusion cage as claimed in claim 1, wherein said activation surface is an inclined surface, and said sliding member has a surface to be in surface contact with said activation surface.

7. The spine fusion cage as claimed in claim 1, wherein an upper surface of said movable member and a lower surface of said housing are provided with skidproof structures.

8. The spine fusion cage as claimed in claim 1, wherein said housing, said movable member, said sliding member and/or said screw are made by biocompatible material.

9. The spine fusion cage as claimed in claim 1, wherein said housing, said movable member, said sliding member and/or said screw are made by Ti, Ti alloy or Polyetheretherketone (PEEK).

10. The spine fusion cage as claimed in claim 1, wherein said activation surface is a curved surface or an inclined surface.
